(19) United States
(12) Patent Application Publication
(10) Pub. No.: US 2020/0107795 A1
(43) Pub. Date: Apr. 9, 2020

(54) REAL-TIME DIAGNOSTIC IMAGING PREVIEW

(71) Applicant: KONINKLIJKE PHILIPS N.V., EINDHOVEN (NL)

(72) Inventors: SHLOMO GOTMAN, HAIFA (IL); KATRINA READ, PHOENIX, MD (US)

(21) Appl. No.: 16/500,844

(22) PCT Filed: Mar. 30, 2018
(86) PCT No.: PCT/EP2018/058276

§ 371 Date: Oct. 4, 2019

Related U.S. Application Data
(60) Provisional application No. 62/486,104, filed on Apr. 17, 2017.

(51) Int. Cl. A61B 6/00; A61B 6/03; G06T 7/00

(52) U.S. Cl. CPC A61B 6/5223; A61B 6/464; A61B 6/463; G06T 2211/428; A61B 6/4405; A61B 6/032; G06T 7/0012; A61B 6/563

(57) ABSTRACT

A system (100) includes an imaging system (102) and a dedicated image review station computing system (104). The imaging system includes an acquisition system (112 and 114) and a reconstructor (116). The dedicated image review station computing system include at least one display monitor (124), a processor (128), and memory (130) with a dedicated review station instruction. A communication channel (136) is between the imaging system and the dedicated image review computing system. The imaging system is configured to, in response to reconstructing an image with the reconstructor with data acquired by the acquisition system, automatically transmit, via the communication channel, the image to the dedicated image review computing system, which is configured to independently display selected images of a set of received images from the imaging system respectively in at least one view port generated with dedicated review station instruction executed by the processor and displayed in the at least one display monitor.

### FIELD OF THE INVENTION

The following generally relates to imaging and more particularly to a real-time diagnostic imaging preview, e.g., for trauma/emergency and/or other situations, and is described with particular application to computed tomography (CT), but is also amenable to other imaging modalities such a magnetic resonance imaging (MRI), digital X-ray, etc.

### BACKGROUND OF THE INVENTION

A CT scanner has been used as a primary imaging modality in various emergency scenarios such as trauma, stroke, cardiac emergencies, etc. In such scenarios, the time to perform primary diagnostic reading of the acquired images becomes critical and is often a life-saving factor. This means that ideally the medical personnel should be able to review the images as soon as they are generated by the CT scanner. These personnel may include a radiologist, a neurologist, an anesthesiologist, an orthopedic surgeon, etc. These personnel normally accompany the patient and therefore may be present in the CT room. Generally, there has been at least two approaches to perform the diagnostic reading.

The first approach is similar to reading a regular, non-emergency image. For this approach, the CT scan is performed, and the resulting images are sent to a picture archiving and communication system (PACS) where they are reviewed on a diagnostic display computing system. Generally, the CT scanner and the PACS are on the same network, and both use the digital imaging and communications in medicine (DICOM) standard to store, transmit, etc. image file. In general, the DICOM standard includes a file format and a network communications protocol that uses transmission control protocol/internet protocol (TCP/IP). The second approach includes directly reading images from the CT scanner console as the images are reconstructed and displayed.

With the first approach, unfortunately, significant time is spent to transfer the images to the PACS, and the time depends on the institution network load. The PACS review stations are often located far from CT room, which, in turn, requires extra communication efforts/activities between the scanning team and reading team. With the second approach, unfortunately, the need to look at the images on the CT console conflicts with the needs to perform other scanning activities on the same console such as planning an additional scan, performing different reconstructions, saving images, checking image quality, etc. In addition, different physicians may want to visualize different anatomy and/or utilize a different visualization algorithm. Hence, there is an unresolved need for another approach.

### SUMMARY OF THE INVENTION

Aspects described herein address the above-referenced problems and others.

In one aspect, a system includes an imaging system and a dedicated image review station computing system. The imaging system includes an acquisition system and a reconstructor. The dedicated image review station computing system includes at least one display monitor, a processor, and memory with a dedicated review station instruction. A communication channel is between the imaging system and the dedicated image review computer system. The imaging system is configured to, in response to reconstructing an image with the reconstructor with data acquired by the acquisition system, automatically transmit, via the communication channel, the image to the dedicated image review computing system, which is configured to independently display selected images of a set of received images from the imaging system respectively in at least one view port generated with dedicated review station instruction executed by the processor and displayed in the at least one display monitor.

In another aspect, a computer readable storage medium is encoded with computer readable instructions, which, when executed by a computer processor of a computing system, causes the computer processor to: receive, by a dedicated review station, a set of images generated by an imaging system, wherein each image of the set is automatically transmitted in response to being reconstructed, and display a selected image of the set in at least one view port.

In another aspect, a method includes receiving, at a dedicated review station, a set of images generated by an imaging system. Each image of the set is automatically transmitted in response to being reconstructed. The method further includes displaying a selected image of the set in at least one view port.

Those skilled in the art will recognize still other aspects of the present application upon reading and understanding the attached description.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an example system including an imaging system and a computing system with a dedicated review station instruction.

FIG. 2 schematically illustrates an example of the dedicated review station instruction.

FIG. 3 schematically illustrates an example of the dedicated review station with a single display monitor with multiple independent view ports and multiple pointing devices.

FIG. 4 schematically illustrates an example of the dedicated review station with multiple display monitors, each with an independent view port.

FIG. 5 schematically illustrates an arrangement of components of the system.

FIG. 6 schematically illustrates another arrangement of components of the system.

FIG. 7 schematically illustrates yet another arrangement of components of the system.

FIG. 8 schematically illustrates a communication channel between the imaging system and the dedicated review station.

FIG. 9 schematically illustrates another communication channel between the imaging system and the dedicated review station.

FIG. 10 schematically illustrates yet another communication channel between the imaging system and the dedicated review station.

FIG. 11 schematically illustrates the dedicated review station in communication with a portable device(s).

FIG. 12 schematically illustrates the dedicated review station instruction with a device interface(s) to a vital sign determining device.

FIG. 13 illustrates an example method in accordance with an embodiment herein.

### DETAILED DESCRIPTION OF EMBODIMENTS

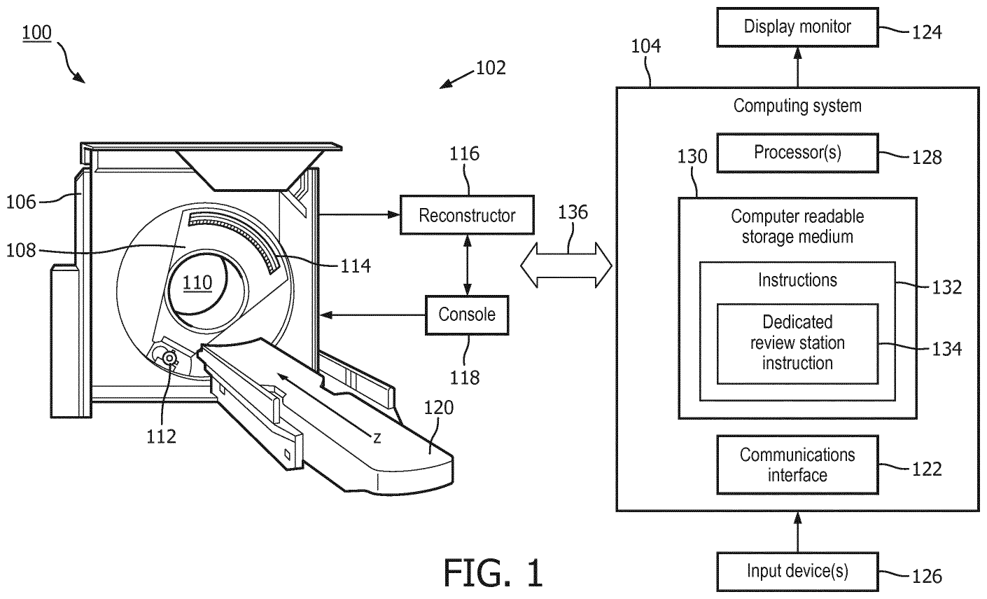

FIG. 1 schematically illustrates a system 100 including an imaging system 102, such as a CT scanner, and a computing system 104 configured at least as a dedicated review station and also referred to herein as the dedicated review station (DRS) and the dedicated review station computing system.

The imaging system 102 includes a generally stationary gantry 106 and a rotating gantry 108, which is rotatably supported by the stationary gantry 106 and rotates around an examination region 110 about a z-axis. A radiation source 112, such as an x-ray tube, is rotatably supported by the rotating gantry 108, rotates with the rotating gantry 108, and emits radiation that traverses the examination region 110. A radiation sensitive detector array 114 subtends an angular arc opposite the radiation source 112 across the examination region 110. The radiation sensitive detector array 114 detects radiation traversing the examination region 110 and generates a signal indicative thereof for each detected photon.

A reconstructor 116 reconstructs the projection data, generating volumetric image data indicative of a scanned portion of a subject or object located in the examination region 110. A general-purpose computing system 104 or computer serves as an operator console. The console 118 includes a human readable output device such as a monitor and an input device such as a keyboard, mouse, etc. Software resident on the console 118 allows the operator to interact with and/or operate the scanner 100 via a graphical user interface (GUI) or otherwise. A subject support 120, such as a couch, supports an object or subject in the examination region 110 before, during and/or after scanning

The dedicated review station 104 includes a communication interface 122, a human readable output device such as a display monitor(s) 124 and an input device(s) 126 such as a keyboard, mouse, a touch screen, etc. The dedicated review station 104 further includes one or more computer processors 128 (e.g., a central processing unit or CPU, a microprocessor, etc.) and computer readable storage medium 130, which excludes transitory medium, such as physical memory, a memory device, and/or other non-transitory storage medium. The computer readable storage medium 130 includes one or more computer readable instructions 132. The one or more computer processors 128 are configured to the execute at least one of the one or more computer readable instructions 132 and/or instructions carried by a carrier wave, a signal and/or other transitory medium.

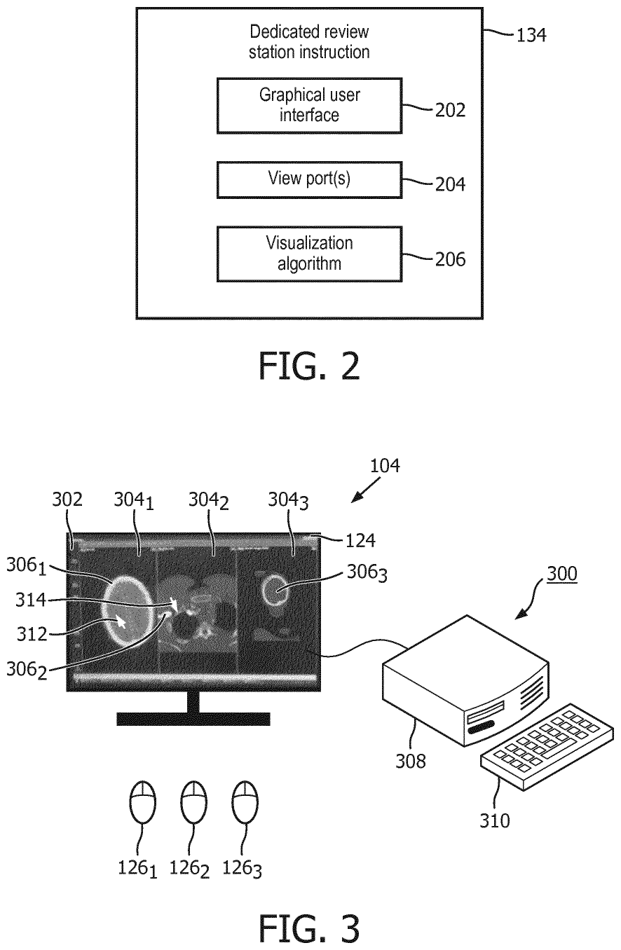

The illustrated computer readable instructions 132 include at least a dedicated review station instruction 134. When executed, the dedicated review station 104 is referred to as a dedicated review station 104. Briefly turning to FIG. 2, in one instance the dedicated review station instruction 134 includes a graphical user interface instruction 202, a view port instruction 204, and a visualization instruction 206. As described in greater detail below, the graphical user interface instruction 202 is configured to render a graphical user interface (GUI) in a single display monitor 124 or different GUIs receptively in different display monitors 124, the view port instruction 204 is configured to render a single view port in each GUI or multiple view ports in each GUI, and the visualization instruction 206 is configured to provide visualization tools (e.g., algorithms) for each view port.

With reference to FIGS. 1 and 2, the dedicated review station 104 receives volumetric image data automatically from the imaging system 102 (and/or other imaging system) as the imaging system 102 acquires data and generates the volumetric image data during a scan. This data is referred to as “real-time” data herein at least since the data is transferred and available as soon the data is generated. As described in greater detail below, the real-time data can be automatically transferred via a channel 136, which may include a healthcare facility/imaging center network and/or other computer network, and/or a dedicated and/or direct connection there between, without an intermediate system such as a PACS and/or otherwise having to run a software application, select an image file, e.g. The system 100 can be pre-configured to transfer all or less than all of the images.

In one instance, the dedicated review station 104 provides immediate access to the received images. Immediate, as used here, refers to as soon as the dedicated review station 104 receives an image. For instance, if a scanned series contains 1000 images being transferred (which can take several dozens of seconds, depending on reconstruction speed), the user can start scrolling through a subset of what has been already received, and once new images arrive, the scroll operation covers more and more images. Additionally or alternatively, the dedicated review station 104 allows, concurrently and independently, different personnel to visualize (including manipulate) the same or different anatomy from these images. This may allow multiple diagnoses and/or decisions to be made simultaneously. Furthermore, the dedicated review station 104 mitigates interruption of workflow as the console 118 can be used to plan and/or scan patients independent of any visualization at the dedicated review station 104.

Turning to FIG. 3, an example of the (computing system) dedicated review station 104 is schematically illustrated. In this example, the dedicated review station 104 includes a computer 300 configured as a desktop 308 and including a wireless keyboard 310, a single display monitor 124, and multiple pointing devices 1261, 1262 and 1263. The computer 300 could alternatively be a tower, a workstation, a laptop, a tablet, and/or other computer. An example operating system that supports multiple pointing devices is Windows® operating system, a product of Microsoft, a company headquartered in Wash., USA. Additionally or alternatively, the dedicated review station 104 includes multiple device drivers which support the multiple pointing devices 1261, 1262 and 1263.

Executing the dedicated review station instruction 134 causes the processor 128 to display of a single graphical user interface 302 in the single display monitor 124. The graphical user interface 302 includes separate view ports 304₁, 304₂ and 304₃, each configured to display a single image 306₁, 306₂ and 306₃, and each controlled with a wireless mouse pointing device 126₁, 126₂ and 126₃. In a variation, at least one of the pointing devices 126₁, 126₂ and 126₃ can be wired and/or another type of pointing device such as a track ball, a touchpad, a digital pen, etc. Likewise, the keyboard can alternatively be wired. Furthermore, in other embodiments, the graphical user interface 302 includes more or less view ports (e.g., only one, two, or more than three), and the dedicated review station 104 includes more or less pointing devices input devices 126.

Each of the view ports 304₁, 304₂ and 304₃ includes its own set of visualization from the visualization instruction 206. For example, each view port independently allows display of a different or same image selected one of the received images, relative to the image(s) displayed in another of the view port(s). Furthermore, each view port includes one or more of the following visualization tools, which can be set independent of another view port: image slice thickness, filter (e.g., bone vs soft tissue), CT number range and center (window/level), slice orientation (e.g., axial, coronal, sagittal and/or oblique), rendering (e.g., 3D (surface, volume, etc.), 4-D, maximum-intensity projection (MIP), minimum-intensity projection, etc.), contrast, brightness, color, segmentation, zoom, pan, rotate, scroll, etc. These actions are not only independent from another view port, but they can also be performed concurrently while performing any of these acts in the other view port.

In one instance, at least one of the pointing devices 126₁, 126₂ and 126₃ can be configured to operate with more than one of the view ports 304₁, 304₂ and 304₃. In this configuration, in one example, the multiple pointing devices 126₁, 126₂ and 126₃ each can be used with each of the view ports 304₁, 304₂ and 304₃. For example, the pointing device 126₁ can be used to open an image in the view ports 304₁ and 304₃, the pointing device 126₂ can be used to set the slice thickness in both of the view ports 304₁ and 304₃, and the pointing device 126₃ can be used to point, alternatively, in any one of the view port 304₁, 304₂ and 304₃. In a variation, each of the pointing devices 126₁, 126₂ and 126₃ is dedicated to a single one of the view ports 304₁, 304₂ and 304₃. FIG. 3 shows a first pointer 312 in the view port 304₁ controlled by the pointing devices 126₁, and a second different pointer 314 in the view port 304₂ controlled by the pointing devices 126₁, which can be used simultaneously or serially. It is to be understood that the size and shape of the first pointer 312 and the second pointer 314 is for explanatory purposes and is not limiting.

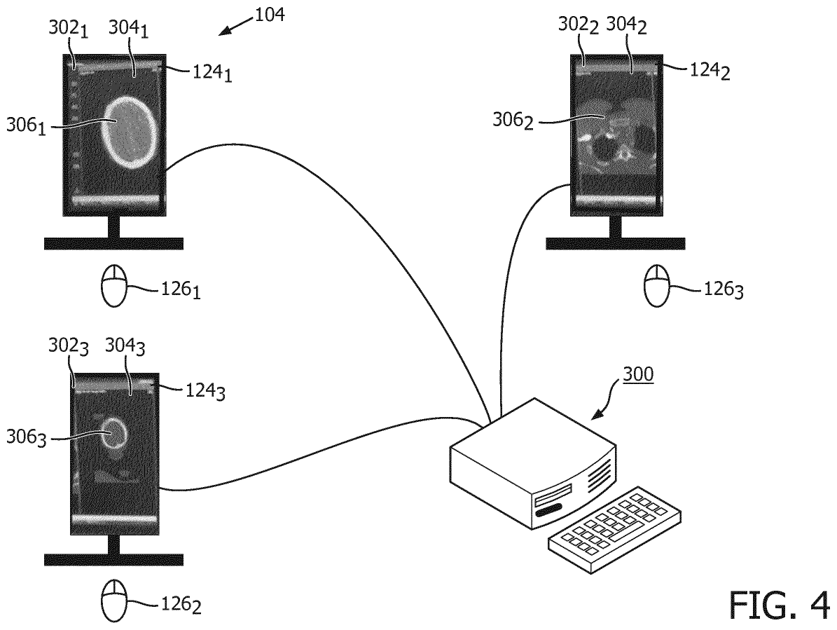

FIG. 4 schematically illustrates a variation of the configuration described in connection with FIG. 3 in which the display monitor 124 includes multiple display monitors 124₁, 124₂, and 124₃, each respectively including its own GUI 302₁, 302₂, and 302₃, each respectively with its own one of the view ports 304₁, 304₂ and 304₃, each respectively displaying one of the images 306₁, 306₂ and 306₃. In a variation, the dedicated review station 104 includes more or less monitors 124. In another variation, at least one of the monitors 124₁, 124₂, and 124₃ includes a GUI with more than one view port, such as a configuration combining the configurations described in FIGS. 3 and 4.

With FIGS. 3 and/or 4, in one instance, at least two of the pointing devices 126₁, 126₂ and 126₃ can be operated simultaneously or serially respectively by different users. This includes a first user operating one of the at least two of the pointing devices 126₁, 126₂ and 126₃ simultaneously with another user operating a different one of the at least two of the pointing devices 126₁, 126₂ and 126₃. The simultaneous operation can be in a same view port and/or different view ports. In another instance, one or more of the pointing devices 126₁, 126₂ and 126₃ can be operated simultaneously or serially by a single user. This includes simultaneously operating more than one of the pointing devices 126₁, 126₂ and 126₃ in a same view port and/or different view ports.

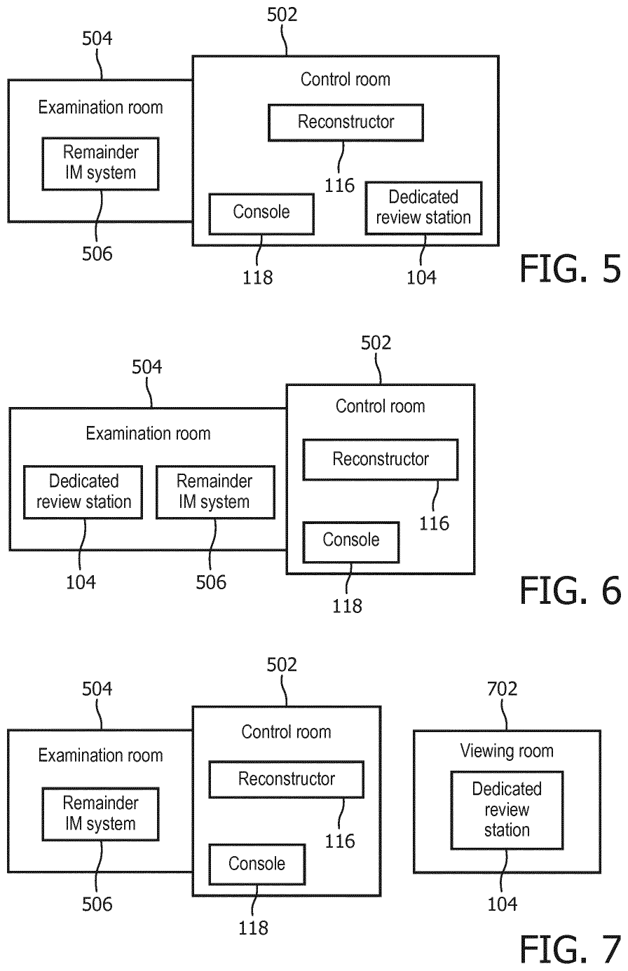

FIG. 5 schematically illustrates an example in which the dedicated review station 104, the reconstructor 116 and the console 118 are located in a control room 502, and a remainder 506 (components 102-114) of the imaging system 102 (REMAINDER IM SYSTEM) are located in an examination room 504, which is a separate and distinct room relative to the control room 502. In this example, the examination room 504 is the room in which a patient is scanned and includes a material in the walls, etc., which absorbs, blocks, attenuates, etc. radiation, mitigating radiation from leaving the examination room 504. It is to be understood that the size and shape of the rooms 502 and 504 are for illustrative purposes and is not limiting. Generally, the rooms 502 and 504 are next to each other with a lead glass and/or other window there between.

FIG. 6 schematically illustrates a variation in which the reconstructor 116 and the console 118 are located in the control room 502, and the remainder 506 of the imaging system 102 and the dedicated review station 104 are located in the examination room 504.

FIG. 7 schematically illustrates another variation in which the reconstructor 116 and the console 118 are located in the control room 502, the remainder 506 of the imaging system 102 is located in the examination room 504, and the dedicated review station 104 is located in another or viewing room 702, which is a separate and distinct room relative to the control room 502 and the examination room 504. In FIG. 7, the viewing room 702 is remote from the rooms 502 and 504. In a variation, the viewing room 702 can share a wall(s) with one or both of the rooms 502 and 504.

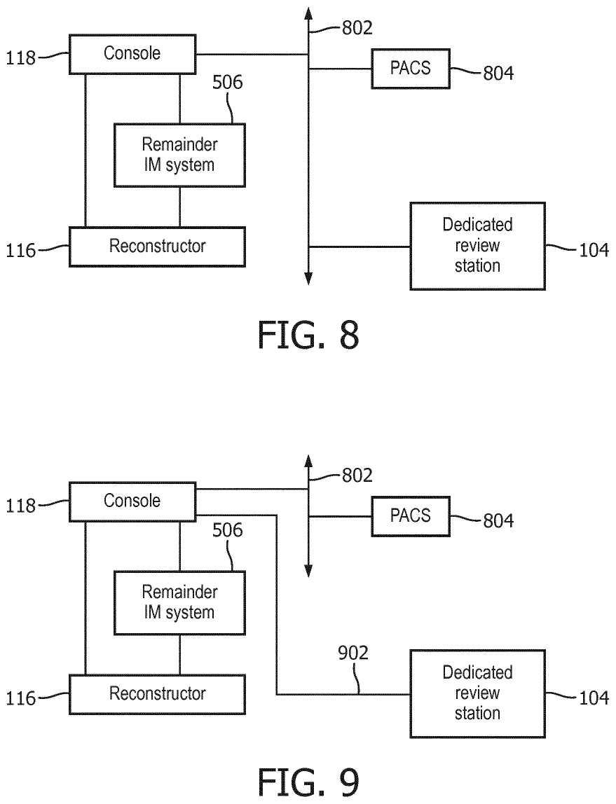

FIGS. 8, 9 and 10 schematically illustrate examples of the communication channel 136 between the imaging system 102 and the dedicated review station 104.

In FIG. 8, the console 118 and the dedicated review station 104 are both connected to a common network 802, such as one part of a radiology information system (RIS), a hospital information system (HIS), etc. A PACS 804 is also shown connected to the common network 802. In a variation, the PACS 804 is omitted. In this example, the console 118 and the dedicated review station 104 (and the PACS 804) are all configured to save, receive and/or transfer images using the DICOM standard. Furthermore, the console 118 and the dedicated review station 104 communicate with each other over the common network 802. The console 118, once an image is generated by the reconstructor 116, automatically transfers the image to the dedicated review station 104 via the DICOM standard over the common network 802.

FIG. 9 schematically illustrates a variation of the system described in connection with FIG. 8. The console 118 and the PACS 804 are likewise connected to the common network 802. In this variation, the console 118 and the dedicated review station 104 are connected via a dedicated connection 902 there between. In one instance, the dedicated connection 902 is a dedicated network cable, and communication there between is independent of the bandwidth of the network 802. The console 118, once an image is generated by the reconstructor 116, transfers the image to the dedicated review station 104 via the DICOM standard over the dedicated connection 902. This approach may transfer image files over quicker than the approach described in connection with FIG. 8 since the connection 902 is a dedicated connection.

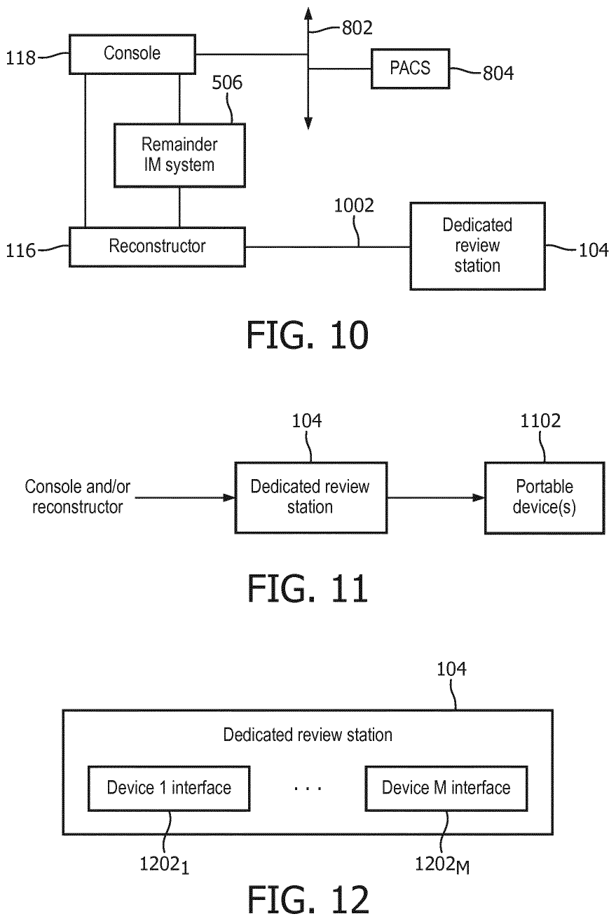

FIG. 10 schematically illustrates another variation of the system described in connection with FIG. 8. The console 118 and the PACS 804 are likewise connected to the common network 802. In this variation, the reconstructor 116 and the dedicated review station 104 are connected via a direct connection 1002 there between. The reconstructor 116 is configured to automatically transfer images directly to the dedicated review station 104 using a non-DICOM protocol, such as a proprietary protocol and/or other protocol. The direct connection 1002 can be wired and/or wireless, and the data link can be point-to-point, Ethernet, etc. This approach may transfer image files over quicker than the approach described in connection with FIGS. 8 and 9 since the images are not converted into and/or from the DICOM format.

FIG. 11 schematically illustrates another variation where the dedicated review station 104 is also in communication with a portable device(s) 1102 such as a smartphone, a laptop, a tablet, etc. In one instance, the dedicated review station 104 transfers screen copy of what is displayed on the dedicated review station 104 to the portable device(s) 1102. In this instance, the screen copy mirrors what is displayed on the dedicated review station 104. In another instance, the dedicated review station 104 transfers the same set of images currently shown via the dedicated review station 104, and the portable device(s) 1202 includes an application to display an image and manipulate it via at least a subset of the functions provided by a visualization algorithm, as described herein and/or otherwise. Additionally or alternatively, the images are transferred directly from the console 118 and/or the reconstructor 116 to the portable device(s) 1102.

FIG. 12 illustrates a variation in which the dedicated review station 104 further includes one or more device interfaces 1202₁, . . . , 1202_{M}, where M is an integer greater or equal to one. Examples of interfaces include but are limited to at least one physiological sensing device such as a blood pressure (BP) device interface, an electrocardiogram (ECG) interface, a pulse oximetry (SO₂) device interface, a heart rate monitor, and/or other device the measures, senses, and/or derives a vital sign. In this variation, the data received (e.g., a signal indicative of a vital sign, etc.) from one or more corresponding devices through the one or more device interfaces 1202₁, . . . , 1202_{M}, is also displayed via the dedicated review station 104.

In another variation, the dedicated review station 104 includes and/or is in communication therewith a computing system including computer-assisted diagnostic (CADx) system, computer-assisted detection (CADe), and/or the like. In general, CADx and CADe systems are configured to assist a clinician with interpreting a medical image. In one instance, this includes marking conspicuous structures and sections and/or evaluating conspicuous structures.

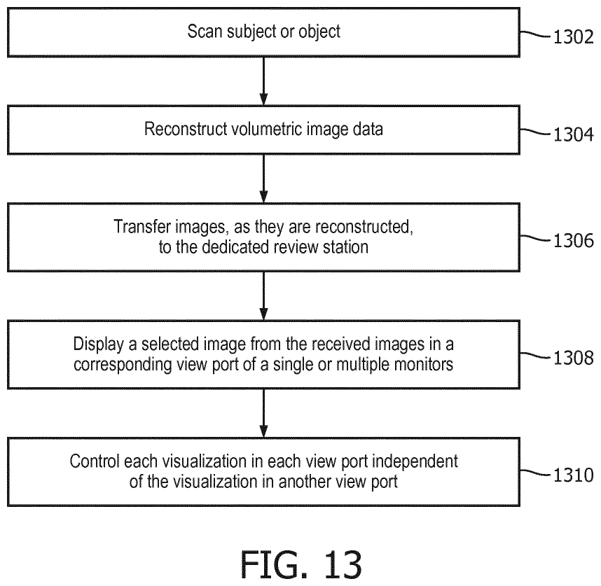

FIG. 13 illustrates an example method in accordance with an embodiment herein.

It is to be appreciated that the ordering of the above acts is not limiting. As such, other orderings are contemplated herein. In addition, one or more acts may be omitted and/or one or more additional acts may be included.

At 1302, a scan of an object or subject is performed.

At 1304, volumetric image data is generated from the acquired data.

At 1306, images of the volumetric image data are automatically transferred to the dedicated review station 104 as they are generated, as described herein and/or otherwise.

At 1308, received images are displayed by the dedicated review station 104 in independent view ports, as described herein and/or otherwise.

At 1310, displayed images are visualized independent of each other.

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium, which is not computer readable storage medium.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A system, comprising:
an imaging system, including:
an acquisition system; and
a reconstructor;
a dedicated image review station computing system, comprising:
at least one display monitor;
a processor; and
memory with a dedicated review station instruction; and
a communication channel between the imaging system and the dedicated image review computing system, wherein the imaging system is configured to, in response to reconstructing an image with the reconstructor with data acquired by the acquisition system, automatically transmit, via the communication channel, the image to the dedicated image review computing system, which is configured to independently display selected images of a set of received images from the imaging system in at least one view port generated with the dedicated review station instruction executed by the processor and displayed on the at least one display monitor.

2. The system of claim 1, further comprising at least two view ports, and wherein a single display monitor displays the at least two view ports, each independently displaying an image of the set of received images.

3. The system of claim 1, further comprising at least two view ports multiple display monitors, each monitor displaying at least one view port of the at least two view ports, and each view port independently displaying an image of the set of received images.

4. The system of claim 1, further comprising at least two view ports and multiple display monitors, at least one display monitor displaying multiple view ports, each view port independently displaying an image of the set of received images.

5. The system of claim 2, wherein each view port includes a set of visualization tools, which act only on the image in the corresponding view port.

6. The system of claim 2, wherein the dedicated image review computing system further comprises multiple pointing devices each pointing device configured to operate with only a single view port.

7. The system of claim 2, wherein the dedicated image review computing system further comprises multiple pointing devices, at least one pointing device configured to operate with multiple view ports.

8. The system of claim 1, wherein the communication channel includes a network, and the imaging system includes a console, and wherein the console and the dedicated image review computing system are in communication with the network, and the console is configured to automatically transfer images to the dedicated image review computing system over the network.

9. The system of claim 1, wherein the imaging system includes a console, and the communication channel includes a dedicated connection between the console and the dedicated image review computing system, and the console is configured to automatically transfer images to the dedicated image review computing system over the dedicated connection.

10. The system of claim 8, wherein the console is configured to transfer images formatted in a digital imaging and communications in medicine file format over the network.

11. The system of claim 1, further comprising=a direct connection between the reconstructor and the dedicated image review computing system, and the reconstructor is configured to automatically transfer images directly to the dedicated image review computing system over the direct connection.

12. The system of claim 11, wherein the reconstructor is configured to transfer images in a non-digital imaging and communications in medicine file format over the direct connection.

13. The system of claim 1, wherein the dedicated image review computing system is configured to transfer received images to a portable device.

14. The system of claim 1, wherein the dedicated image review computing system further includes at least one device interface to a physiological sensing device, and the dedicated image review computing system is configured to display a physiological value received through the at least one device interface.

15. A non-transitory computer readable storage medium encoded with computer readable instructions which, when executed by at least one processor, cause the at least one processor to perform a method comprising:
receiving, by a dedicated review station, a set of images generated by an imaging system, wherein each image of the set is automatically transmitted in response to being reconstructed; and
displaying a selected image of the set in at least one view port.

16. The non-transitory computer readable storage medium of claim 15, further comprising providing at least two view ports in a same graphical user interface of a same display monitor, and the at least one processor independently and concurrently visualizes a first selected image in a first of the view ports and a second selected image in a second of the view ports.

17. The non-transitory computer readable storage medium of claim 15, further comprising providing at least two view ports, each view port displayed on a different display monitor, and wherein a first image is displayed in a first view port displayed in a first display monitor, and a second image is displayed in a second view port displayed in a second display monitor.

18. A method, comprising:
receiving, at a dedicated review station, a set of images generated by an imaging system, wherein each image of the set is automatically transmitted in response to being reconstructed; and
displaying a selected image of the set respectively in at least one view port.

19. The method of claim 18, further comprising providing at least two view ports in a same graphical user interface of a same display monitor, and independently and concurrently visualizing a first selected image in a first of the view ports and a second selected image in a second of the view ports.

20. The method of claim 18, further comprising providing at least two view ports, each view port displayed on display monitor, and wherein a first image is displayed in a first view port displayed on a first display monitor, and a second image is displayed in a second view port displayed on a second display monitor.
